(19) United States
(12) Patent Application Publication
(10) Pub. No.: US 2008/0109004 A1
(43) Pub. Date: May 8, 2008

(54) Invasive Distracter

(76) Inventors: Orlando Da Rold, Solothurn (CH); Hassan Sadri, Riaz (CH)

Correspondence Address: Calvin G Covell;Tarolli Sundheim Covell & Tummino, 1300 East Ninth Street, Suite 1700, Cleveland, OH, 44114, US

(21) Appl. No.: 11/597,123

(22) PCT Filed: May 11, 2005
(86) PCT No.: PCT/IB05/51535

§ 371 Date: Nov. 5, 2007

(30) Foreign Application Priority Data
May 25, 2004 (CH) 00896/04

(51) Int. Cl. A61B 17/58

(52) U.S. Cl. 606/99

(57) ABSTRACT

An invasive distractor is proposed that has a form comparable to a conventional C-clamp or screw clamp. It permits spatially targeted positioning of two bones, so that the joint connecting these bones can be spread by means of distraction during an intervention, thereby creating the space needed to perform diagnosis and/or interventions by means of arthroscopic methods. The appliance is composed of a base (2) and of a slide (3) which can be displaced relative to one another in a controlled manner and secure against twisting. The base (2) and the slide (3) comprise various devices, such as worm gear, rotary gear and expansion gear, to ensure that the distraction of the two bones can be performed in a spatially exact manner adapted to the particular requirements.

### FIELD OF THE INVENTION

The present invention relates to an invasive distractor.

### BACKGROUND OF THE INVENTION

The methods of distraction and compression of bones are widely known in orthopaedics. In surgery of the jaw, these methods are used to correct the positions of the teeth and jaw. In cases of angle defects or length defects caused by congenital malformations or by incorrectly fused bone fractures, these methods are applied using the appropriate devices. Devices for corrections of individual bones are described, for example, in EP 0 699 419 A and in EP 0 858 781 A. Such devices are also used in accident and emergency surgery. They are used mainly as so-called fixators, in order to bring the two parts of a broken bone into the correct position for fusion and also to hold them in this position.

Another use, to which the present invention relates, is for the distraction of joints. To treat tissues in proximity to joints and within joints, distraction is often required for surgical interventions. The two bones adjoining the joint are spread so far apart as to make possible the desired intervention or arthroscopic examination of the joint. Depending on the treatment, it is necessary to alter distances and angles during the distraction of joints. In each case, a device has to ensure that the two bones can be held securely in the intended position.

The force that has to be applied for distraction of a joint depends on the muscles, ligaments, tendons and nerve strands that have to be stretched. Care must be taken to ensure that a joint is never overextended. Estimating how far extension should go is difficult, and it may differ from case to case. Not only is every joint different, the sensitivity of each patient is also different. For example, it has been found that a joint that has already been subjected several times before to a surgical procedure behaves differently than a joint on which an operation is being performed for the first time.

Controlled distraction permits arthroscopy. Arthroscopy procedures, and interventions in joints carried out under arthroscopic control, are gaining in popularity, and this type of intervention is less risky than an operation performed on an open wound. In many cases, minimally invasive interventions of this kind do not require a hospital stay, and the patient is mobile within a day. The patient comes to the hospital for the operation and can return home that evening. This is only possible because the risk of infections is much lower than in an operation performed on an open wound. It is also advantageous in terms of health service costs. For interventions of this kind performed on the ankle joint, Patent Application No. WO 96/00529 has disclosed a device that permits controlled and precise distraction.

For many interventions, for example in the knee joint, bending the joint into the correct position is often sufficient for arthrosopy and/or minor interventions on the tissue. Many arthroscopy procedures performed on the knee joint are carried out in this way. For the hip joint, however, this is difficult, because the hip joint is difficult to access.

Various methods are used for distraction of a joint. To perform distraction of the shoulder joint, for example, a weight is suspended from the arm, and this pulls the arm down and in this way distracts the shoulder joint.

The same method is used, with correspondingly greater weights, on the knee joint, ankle joint and hip joint. Such relatively simple devices are not especially suitable for this application and have considerable disadvantages. On the one hand, the force applied for extension always depends on how the suspended weight acts on the body part. The angle at which the weight is intended to pull is of critical importance. At the same time, this direction in which the weight is intended to pull, and which is determined by the operating surgeon, is often not optimal for the set-up in the operating theatre. The operating surgeon, assistants and theatre nurses have to move around the device. Making contact with the device is problematic, because every movement thereof causes a change in the distraction of the joint.

An intervention on the hip joint is especially difficult. The force that has to be used in order to distract the joint and that is applied to the foot is considerable. The positioning, i.e. distraction of the joint, within a defined range is difficult. A hip joint must in principle be distracted completely. A further disadvantage lies in the side effect of performing distraction by pulling on the foot: nerves, ligaments, tendons and muscles are stretched too. In many cases it has been found that the nerves do not tolerate excessive stretching. Patients have complained that the feeling in their legs is no longer as it should be. It is suspected that this impairment is due to overstretching of the sciatic nerve. For hip joint operations, orthopaedic surgeons have therefore preferred an open operation rather than an arthroscopic intervention by means of distraction.

Overstretching of the tissues is mainly associated with the difficulty involved in precisely setting the force with which extension is to be applied to the joint that is to be operated on. In normal cases, this is down to the feel of the orthopaedic surgeon carrying out the treatment. In many operations performed by means of simple invasive distractors, which allow the force to be set but do not permit control of the position, experience has been gathered on treating hip joints by this method. No patient is known of who has complained of the side effects described above.

The great advantage of this method are the small interventions that are needed. The patients leave the operating theatre with a small number of incisions that close rapidly. Moreover, the risk of infection in this method is very much lower than in an operation performed on the open hip joint.

Known fixators and distractors, as are described in many patents, have the disadvantage that their main axis and the actual support of the force with which the distraction is to be performed is arranged close to and parallel with the limb that is to be distracted, the reason being that these distractors are provided for positioning in bone fracture cases and for lengthening of individual bones.

For treatment of hip joints, devices of this kind also impede the arthroscopic intervention. They obstruct the operating field accessible to the operating surgeon. Moreover, although they allow the hip joint to be spread, changes in position for bringing femur and pelvis into another angle relative to one another are not possible. Moreover, because all the existing distractors are not intended for spreading a hip joint, the base bar or support bar is in the way of images being taken by the camera. However, arthroscopy, that is to say a procedure for looking into the joint, is an absolute prerequisite if the objective is to remove tissue from the interior of a joint.

### SUMMARY OF THE INVENTION

The object of the present invention is to improve an invasive distractor of the type mentioned above in such a way that the advantages of the known distractors are maintained. The appliance makes it possible to offer the operating surgeon a free working area and is also not in the best area for the imaging by the X-ray camera, and it also allows the relative angle of the two bones forming the joint to be changed rapidly and with precision during the intervention, and the operating surgeon can at all times control the applied force necessary for the distraction.

This object is achieved by an invasive distractor having the features of the present invention. Further features and their advantages are explained in the description below.

### BRIEF DESCRIPTION OF THE DRAWINGS

In the drawing:

FIG. 1 is a perspective view of the invasive distractor,

FIG. 2 is a perspective drawing of the invasive distractor,

FIG. 3 is a view of the invasive distractor,

FIG. 4 is a view of the base of the invasive distractor,

FIG. 5 is a view of the slide of the invasive distractor,

FIG. 6 is a detail of the threaded bore with guide sleeve,

FIG. 7 is a detail of the threaded bore with drill bushing and drill,

FIG. 8 is a detail of the threaded bore with screw and union nut,

FIG. 9 is a system view showing the positioning of the invasive distractor,

FIG. 10 is a system view showing the arrangement of the screws,

FIG. 11 is a system view showing the arrangement of the screws,

FIG. 12 is a system view showing the arrangement of the screws,

FIG. 13 is a system view showing the arrangement of the screws,

FIG. 14 is a system view showing the arrangement of the screws,

FIG. 15 is a system view showing the arrangement of the screws,

FIG. 16 is a system view showing the arrangement of the screws.

The figures show preferred illustrative embodiments that are explained in more detail in the following description.

### DETAILED DESCRIPTION OF THE PREFERRED EMBODIMENT

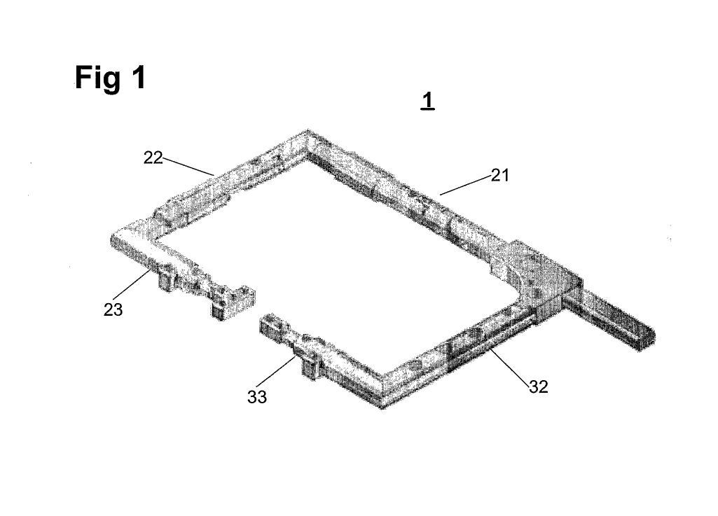

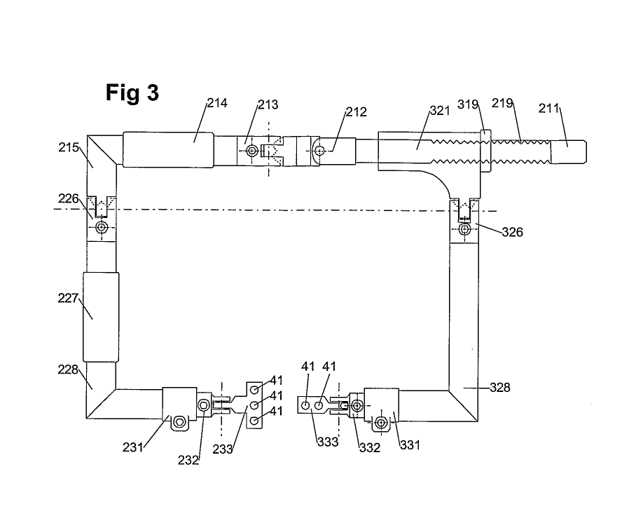

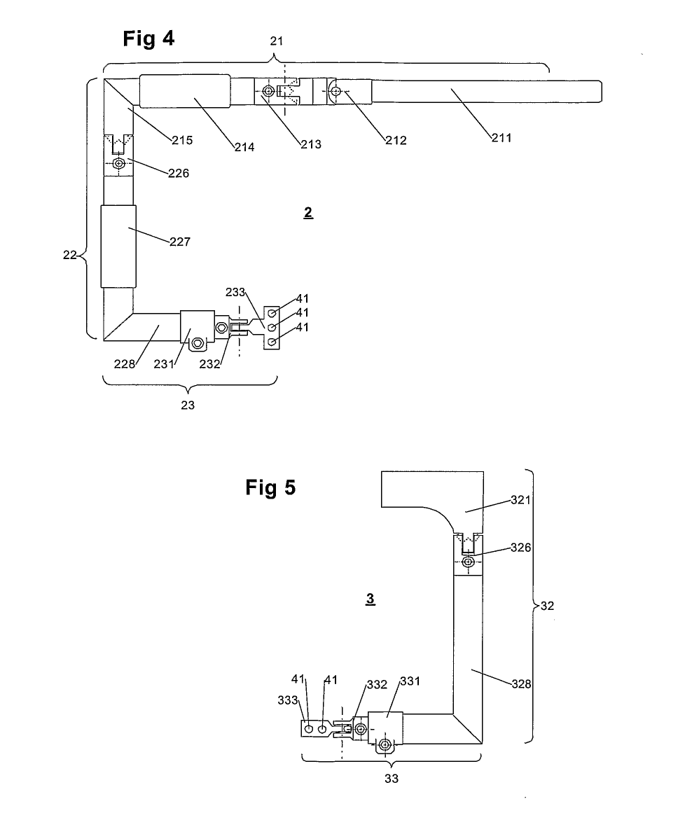

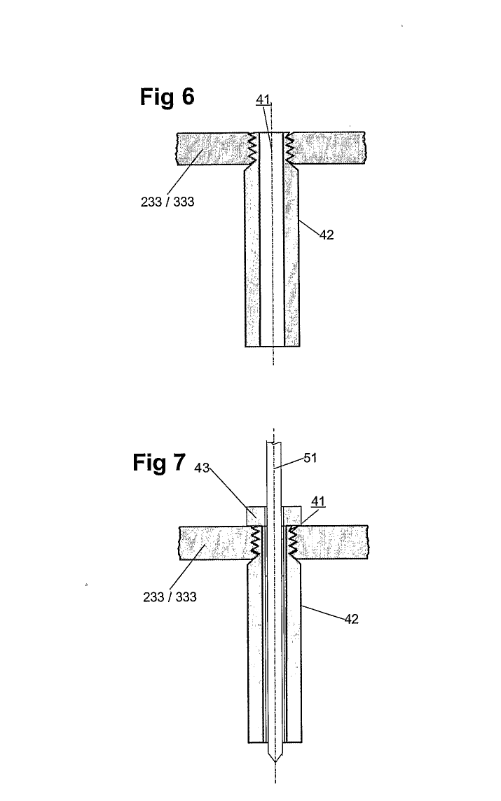

To describe the invasive distractor 1 as fully as possible, the following description deals with the design and mechanical details of the invasive distractor 1 and also with the practical use thereof. The invasive distractor 1 according to the invention consists (FIGS. 1, 2 and 3) of the following parts. The two main components, namely base 2 (FIG. 4) and slide 3 (FIG. 5), are connected to one another (FIG. 3) by the runner 321 of the slide 3 being mounted with a shape fit on the guide part 211 so as to be longitudinally movable thereon and secure against twisting. To be able to control the longitudinal movement, a thread 219 is arranged on the guide part 211. A nut 319 is mounted on the runner 321 so as to be rotatable thereon, but fixedly connected thereto. By turning the nut 319, the latter moves on the thread 219 and, as a result, the runner 321 moves on the guide part 211 by the distance set by the thread pitch and defined by the extent by which the nut 319 is turned.

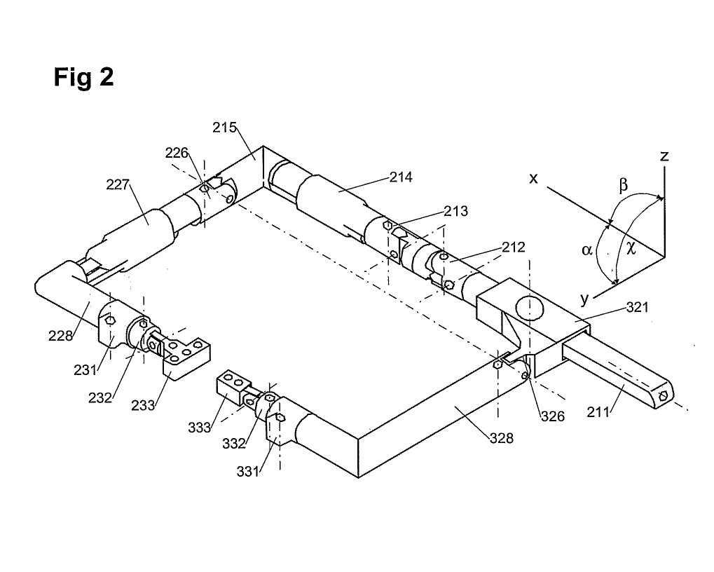

The base 2 (FIG. 4) is composed of a shaft 21, a headpiece 22 and a transverse retainer 23. The shaft 21 is the actual element that permits the distraction itself, while the headpiece 22 and the transverse retainer 23 permit the necessary projection to keep the working space free for the operating surgeon. The shaft 21 is composed of a guide part 211 on which hinges provided with worm gears, axis correction gear X 212 and axis correction gear Z 213, are mounted so as to be movable and free of clearance. The axis correction gear X 212 makes it possible to adapt the angle α (FIG. 2) in the plane x-y between shaft 21 (x-axis) and the headpiece 22 (y-axis). The axis correction gear Z 213 permits adaptation of angle β in the vertical x-z between shaft 21 (x-axis) and headpiece 22 (z-axis).

In addition to the axis correction gear X 212 and the axis correction gear Z, the shaft 21 also comprises a longitudinal axis correction gear 214. The latter serves to lengthen the shaft 22 and, consequently, the base 2 in the angle settings α and β. If, after setting the angles α and β, the distraction at the desired site of the joint is not sufficient or in fact is too great, it can be reset by means of the longitudinal axis correction gear 214. The shaft angle piece 215 is fixed on this longitudinal axis correction gear 214. It connects the shaft 21 to the headpiece 22.

Located on the shaft angle piece 215, as part of the headpiece 22, there is in turn an offsetting gear B 226. With this offsetting gear B 226, the angle χ of the headpiece 22 to the plane y-z can be adjusted. Arranged in the further continuation of the headpiece 22 (FIG. 4), there is a transverse axis correction gear 227 which serves to change the headpiece 22, that is to say the distance between shaft 21 and transverse retainer 23. The head angle piece 228 is fixed on this transverse axis correction gear 227. It connects the headpiece 22 to the transverse retainer 23.

Located on the head angle piece 228, as part of the transverse retainer 23, there is an axial rotation gear pelvis 231. This is used for turning in the plane x-y about angle χ, so that the transverse headpiece 223 can be precisely adapted to the spatial circumstances. This axial rotation gear pelvis 231 is in turn followed by a drop angle gear pelvis 231, with which the transverse headpiece 223 can also be adjusted about angle β to the plane x-y.

The transverse headpiece 233 has three threaded bores 41 with internal threads that can receive corresponding guide sleeves 42. The guide sleeves 42 serve as a guide for the drill bushing 43 and later, in the course of the procedure, for receiving the screws 52. These elements are dealt with in more detail in the description of the operating procedure.

The slide 3 (FIG. 5) is composed of a footpiece 32 and a longitudinal retainer 33. A runner 321 (FIG. 3) is guided on the guide part 211 of the shaft 21. This guide part 211 has an offsetting gear S 326 with which it is possible to adjust an angle χ of the footpiece 32 to the plane y-z. This offsetting gear S 326 is followed by a foot angle piece 328. This connects the footpiece 32 to the longitudinal retainer 33. Located on this foot angle piece, as part of the longitudinal retainer 33, there is an axis rotation gear femur 331. The latter is used for turning in the plane x-y about angle χ, so that the lateral headpiece 333 can be precisely adapted to the spatial circumstances. This axis rotation gear femur 331 is in turn followed by a drop angle gear femur 332 with which the lateral headpiece 333 can also be adjusted by angle β to the plane x-y.

The lateral headpiece 333 has two threaded bores 41 that can receive corresponding guide sleeves 42. The guide sleeves 42 serve as a guide for the drill bushing 43 and later, in the course of the procedure, for receiving the screws 52. These elements are dealt with in more detail in the description of the operating procedure.

An important advantage of the invasive distractor according to the invention and of its use is the possibility of freely adjusting the settings of the screws in the axes x, y and z during the preparatory work, at the start of the operation and even during the actual distraction under application of force, that is to say during the operation.

### Procedure and Method

An intervention performed on a hip joint is described below by way of example:

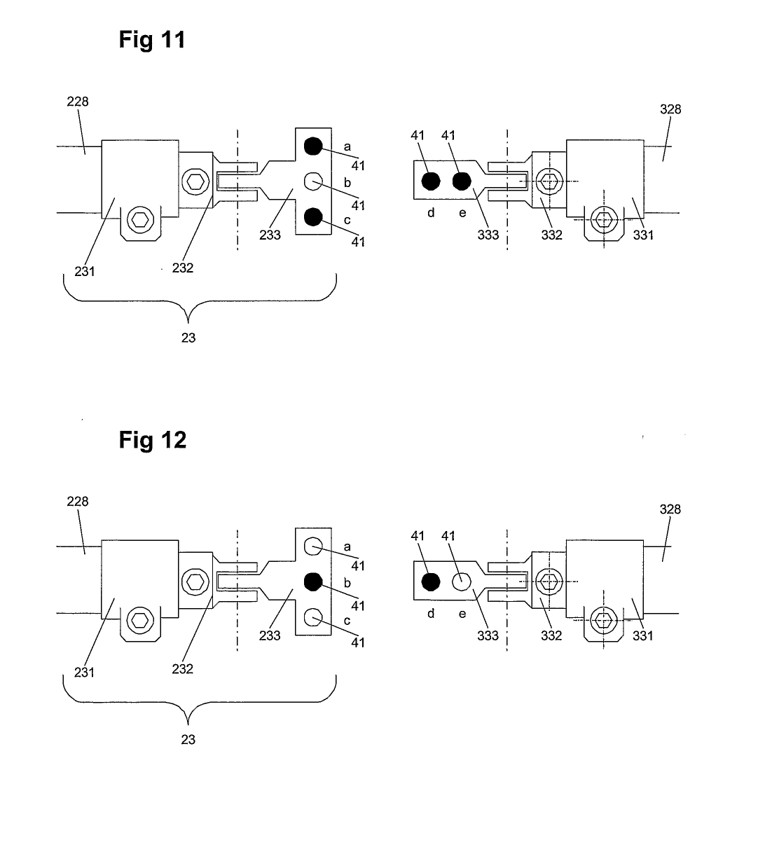

To be able to reliably hold the pelvis and femur side in a hip operation, two screws 51 are whenever possible inserted in the transverse headpiece 233 of the base 2 provided for the pelvis and two screws 51 are inserted in the lateral headpiece 333 of the slide 3 provided for the femur. At least one screw 51 each must be inserted in order to permit distraction. The important advantages of the invasive distractor 1 according to the invention can be best exploited if two screws 51 are inserted in each case. This arrangement of the screws 51 depicted in FIG. 11 is ideal: the two outer positions a and c of the threaded bores 41 are used in the pelvis, and both lateral positions d and e (FIG. 11) of the threaded bores 41 are used in the femur. However, in order to make smaller incisions, the surgeon often chooses the positions ab or bc in the transverse headpiece (FIG. 10).

The invasive distractor 1 is made ready by inserting the desired guide sleeves 42 into the threaded bores 41 provided for them. As has been described above, it is possible, on the pelvis side, that is to say in the transverse headpiece 233, to use one to a maximum of three screws 52 and, on the side of the femur, that is to say in the lateral headpiece 333, to use one to a maximum of two screws 52. The five possibilities are shown in FIGS. 11 to 15. Depending on which side the hip joint to be operated on lies, the femur, that is to say the lateral headpiece 333, and the pelvis, that is to say the transverse headpiece 233, will lie to the left or right.

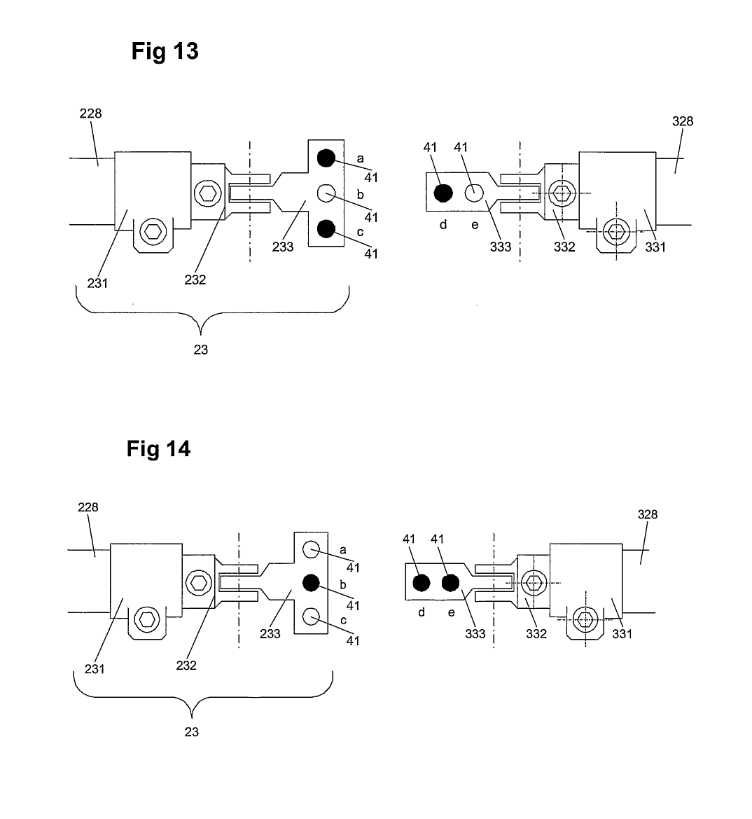

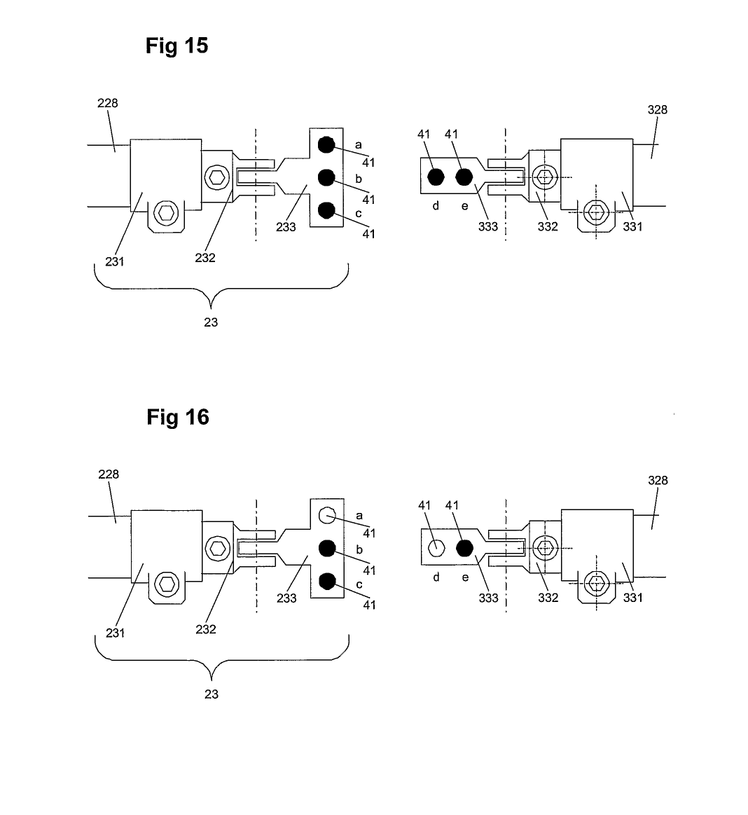

If the positions a and c on the transverse headpiece 233 and both positions d and e on the lateral headpiece 333 can be fitted (FIG. 11), this is ideal. The invasive distractor is connected optimally to pelvis and femur. If at least one screw 52 is fitted in each case (FIG. 12), this represents the absolute minimum. The advantages of the invasive distractor according to the invention are not exploited in this way. The screw has of course to be fitted at position b in the pelvis, or transverse headpiece 233. For the femur, that is to say the lateral headpiece 333, the position of the screw 52 does not play a great role. If two screws 52 can be fitted only on one side (FIG. 13 and FIG. 14), this is satisfactory, but not optimal. For large statures and difficult cases, each position can be used (FIG. 15), but this is done only in rare cases. Instead, it is more common that a further position has to be provided with a screw during the preparatory work and operation.

When the desired guide sleeves 42 are now inserted, the adaptation to the patient is carried out, that is to say the transverse headpiece 233 (FIG. 3) is adjusted by means of axial rotation gear pelvis 231 and drop angle gear pelvis 231 and of the lateral headpiece 333 by means of axis rotation gear femur 331 and drop angle femur 332. After this careful adaptation to the position of the patient, the invasive distractor 1 is placed over the operating site on the surface of the patient's thigh.

An incision is made to prepare a position for a guide sleeve 42 of the pelvis side, and the invasive distractor 1 is then placed onto the patient's pelvis until the inserted guide bushings 42 sit on the patient's bone. A drill bushing 43 (FIG. 7) is then inserted into this guide sleeve 42 and is used to drill a hole in the pelvis by means of the drill 51. The drill bushing 43 is then removed, and the first screw 52 is screwed through this guide sleeve 42 into the bone of the pelvis.

Thereafter, a position is prepared on the femur side by means of an incision, and the guide sleeves 42 are placed on the femoral bone. The femur is drilled on both sides by means of the drill 51 through a drill bushing 43. The drill 51 and the drill bushing 43 are removed, and the screw 52 is screwed into place.

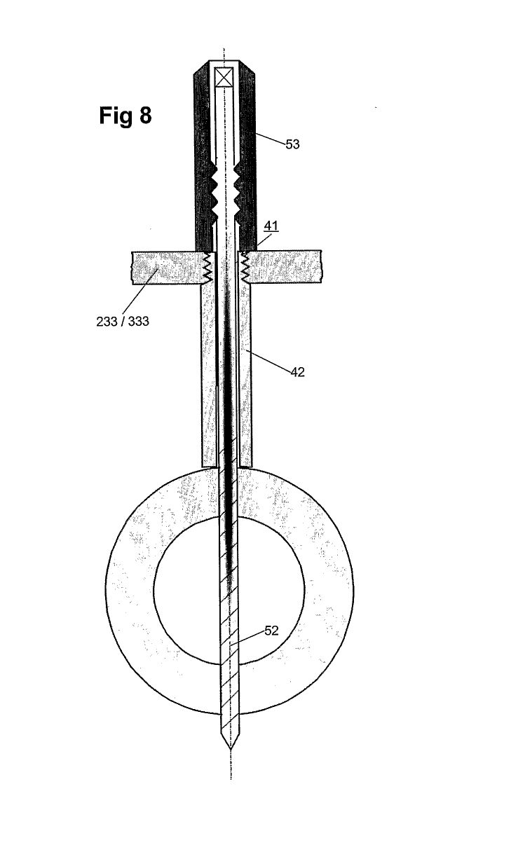

After checking the positions again, a union nut 53 is fitted over each of the screws 52 and tightened (FIG. 8). Tightening of these union nuts 53 on the femur side and pelvis side ensures that the invasive distractor 1 is securely connected to femur and pelvis via two guide sleeves mounted fixedly on the bone. The remaining screws are now screwed in and provided with union nuts 53.

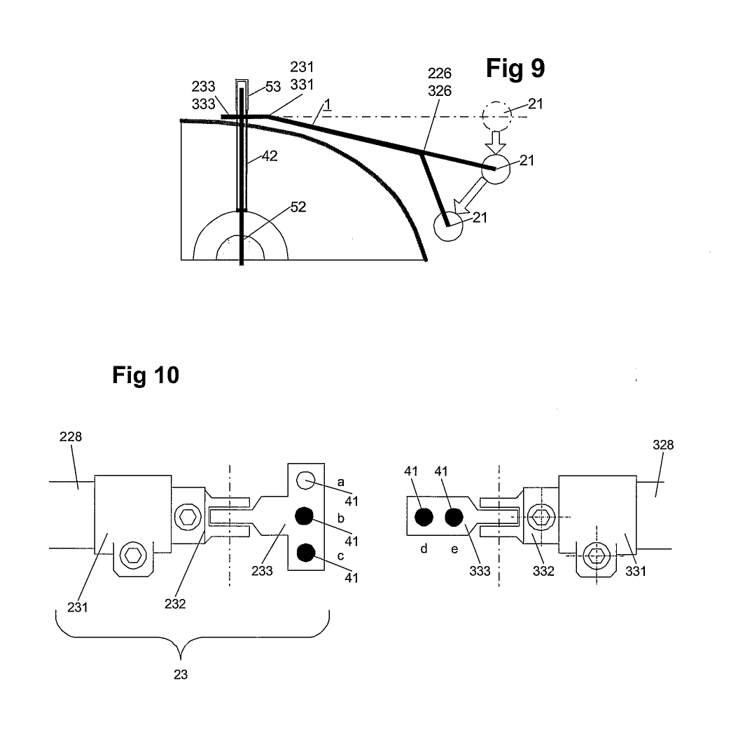

If necessary, the orientation of the invasive distractor 1 from pelvis to femur is now corrected in respect of rotation by means of both axial rotation gear pelvis 231 and axis rotation gear femur 331 (FIG. 3), so that the invasive distractor 1 (FIG. 9) lies on the thigh and pelvis, respectively, of the patient. The shaft 21 is then offset (FIG. 9) by means of offsetting gear B 226 and offsetting gear S 326 (FIG. 3), so that it is not in the way of X-ray images being taken.

Distraction can now be begun. In the guide part 211 there is a threaded rod by means of which the runner 321 can be moved. At the end of the guide part 211, this threaded rod has an internal hexagon with which this threaded rod is turned, and in this way the position of the runner 321 on the guide part 211 can be adjusted. The distance between headpiece 22 and footpiece 32 increases and the distraction of the joint begins. After some time, the capsule is pierced in order to release the vacuum that arises therein. The distraction is continued until the distance between pelvis and femur, that is to say the distance between headpiece 22 and footpiece 32, is large enough for the intended intervention. In some circumstances, the angles have to be reset during the procedure and positions have to be corrected.

The surgical intervention can now be performed. During the arthroscopy work, the invasive distractor 1 may have to be readjusted so that certain locations can be better accessed. Such readjustments can be undertaken in all three directions by the means provided. In the x-axis (FIG. 2) by transverse axis correction gear 227, in the y-axis by longitudinal axis correction gear 214, and in the z-axis by axis correction gear Z 213.

To improve the distraction, arthroscopic capsulotomy can be performed as soon as the surgical accesses are established.

The end of the operation and the removal of the invasive distractor 1 take place in the following sequence:
release of the distraction
loosening of the union nuts 53
detachment of the invasive distractor 1
removal of the screws 52

This description has concentrated on the treatment of the hip joint. A person skilled in the art will appreciate that this invasive distractor 1 can be used for arthroscopy of other joints, for example knee, elbow, ankle, etc. This invasive distractor 1 is also suitable as a fixator for positioning of fractures, for example of the femur, tibia or humerus.

As has been described above, an important advantage of the use of an invasive distractor 1 is the relatively small intervention permitted by it. Recourse to open surgery is possible at any time, should this prove necessary during the intervention.

1. Invasive distractor (1) with a form comparable to a conventional C-clamp or screw clamp, for spatially targeted positioning of two bone parts, so that, during an intervention, it is possible, by means of distraction, to create the necessary space in the joint connecting these bones, thereby permitting arthroscopic methods for diagnosis and interventions, and for repositioning of bone parts after fractures or for correction of growth anomalies, said distractor being composed of a base (2) and of a slide (3), wherein the base (2) and the slide (3) are connected to one another so as to be movable in a controlled longitudinal movement and secure against twisting.

2. Invasive distractor according to claim 1, wherein the base (2) is composed of a shaft (21), a headpiece (22) and a transverse retainer (23).

3. Invasive distractor according to claim 1, wherein the slide (3) is composed of a footpiece (32) and a longitudinal retainer (33).

4. Invasive distractor according to claim 2, wherein the shaft (21) is composed of a guide part (211), an axis correction gear X (212), an axis correction gear Z (213) offset by 90° relative to the latter, a longitudinal axis correction gear (214) and a shaft angle piece (215).

5. Invasive distractor according to claim 2, wherein the headpiece (22) is connected to the shaft (21) via the shaft angle piece (215) and is composed of an offsetting gear B (226), a transverse axis correction gear (227), and a head angle piece (228).

6. Invasive distractor according to claim 2, wherein the transverse retainer (23) is connected to the headpiece (22) via the head angle piece (228) and is composed of an axial rotation gear pelvis (231), a drop angle gear pelvis (232) and a transverse headpiece (233).

7. Invasive distractor according to claim 3, wherein the footpiece (32) is composed of a runner (321), an offsetting gear S (326) and a foot angle piece (328).

8. Invasive distractor according to claim 3, wherein the longitudinal retainer (33) is connected to the footpiece (32) via the foot angle piece (328) and is composed of an axis rotation gear femur (331), a drop angle gear femur (332) and a lateral headpiece (333).

9. Invasive distractor according to claim 4, wherein the guide part (211) is fixedly connected to a thread (219), and the runner (321) is connected to a rotatably mounted nut (319).

10. Invasive distractor according to claim 1, wherein the slide (3), on the side of the footpiece (32) directed towards the base (2), has a runner (321) with which the slide (3) is held in longitudinal position on a guide part (211) of the shaft (21) and is moved along the latter, for which reason a thread (219) is arranged on the guide part (211) and a rotatably mounted nut (319) is connected to the runner (321), the nut (319) being held on the thread (219), and the slide (3) with the runner (311) thus being held in a controlled manner and secure against twisting on the guide part (211) of the base (2) and being able to be moved in a controlled manner along said guide part (211).

11. Operating method using the invasive distractor, characterized by the description in the section headed “Procedure and method”.
